(19) United States
(12) Patent Application Publication
(10) Pub. No.: US 2025/0288298 A1
(43) Pub. Date: Sep. 18, 2025

(54) IMPLANTABLE EMBOLIC MATERIAL DIVERSION DEVICE FOR PREVENTING STROKE AND METHOD OF DELIVERING THE SAME

(71) Applicant: EUREKA Medical Innovation Inc., Jersey City, NJ (US)

(72) Inventor: Boris Leonardo Pabon Guerrero, Medellin (CO)

(21) Appl. No.: 19/079,060

(22) Filed: Mar. 13, 2025

Related U.S. Application Data
(60) Provisional application No. 63/564,634, filed on Mar. 13, 2024.

(51) Int. Cl. A61B 17/12

(52) U.S. Cl. CPC A61B 17/12113

(57) ABSTRACT

An improved implantable embolic material diversion device for preventing stroke and a method of delivering the same are provided. The implantable embolic material diversion device can include a tubular mesh body including a proximal end and a distal end and defining a lumen between the proximal and distal ends. The tubular mesh body can have only a single layer of a plurality of braided wires that form a plurality of openings, each braided wire having a shape memory material. Each opening can be sized to allow a passage therethrough of blood and to prevent the passage therethrough of embolic material having, in every direction of view thereof, at least one straight-line distance between two points on a perimeter of at least one cross-sectional surface in excess of 350 μm.

### CROSS-REFERENCE TO RELATED APPLICATION

This application claims priority to and the benefit of U.S. Provisional Patent Application No. 63/564,634, filed on Mar. 13, 2024, which is hereby incorporated by reference herein in its entirety for all purposes.

### TECHNICAL FIELD

In general, various embodiments of this invention relate to an implantable embolic material diversion device and, more specifically, to an implantable embolic material diversion device for diverting embolic material to prevent ischemic stroke and a method of delivering the same to a multifurcated zone in a patient's body.

### BACKGROUND

In general, two arteries, referred to as common carotid arteries (CCA), supply a major portion of blood supply to the brain hemispheres. Each of the CCAs branches off (e.g., bifurcates) into an internal carotid artery (ICA) and an external carotid artery (ECA). Further, two vertebral arteries each supply blood to the brain stem. Thus, the ICAs and vertebral arteries supply blood to subsequent intracranial blood vessels of the brain.

A stroke refers an abrupt impairment of brain function caused by pathologic changes occurring in blood vessels. The main cause of strokes is insufficient blood flow to the brain (referred to herein as an “ischemic stroke”). Ischemic strokes are caused by sudden occlusion or partial occlusion of a blood vessel (e.g., an artery) supplying blood to the brain, which can result from embolic material forming in the bloodstream and flowing into carotid and vertebral arteries leading towards the brain. Some non-limiting examples of embolic material can include blood clots, air bubbles, and fat globules.

To prevent ischemic strokes resulting from embolic material occluding a blood vessel supplying blood to the brain, conventional blood clot diversion devices have been contemplated for placement in a multifurcated zone in which a main blood vessel branches into at least two branch blood vessels. Such devices could function to deflect and divert embolic material (e.g., blood clots) originating from a main blood vessel away from a particular branch blood vessel toward another different branch blood vessel leading to a different part of the circulatory system, while causing an insignificant change to blood flow for the branch blood vessel for which embolic material is diverted. For example, conventional blood clot diversion devices have been contemplated for placement between the CCA and ECA to deflect and divert embolic material from entering the ICA and intracranial circulatory system, thereby preventing such embolic material from causing occlusion of the ICA or a subsequent intracranial blood vessel of the intracranial circulation system that is coupled to the ICA. However, such devices suffer from having openings that fail to filter and divert fine embolic material and allow the fine embolic material to flow into the branch blood vessel for which embolic material diversion is desired, such as a branch blood vessel that supplies blood to the brain.

Further, while devices have been introduced that include openings small enough to filter fine embolic material, such devices conventionally restrict and prevent re-access and re-crossing to the branch blood vessel for which the device diverts embolic material. In the event of a medical emergency requiring access that blood vessel and/or to the blood vessels extending beyond that blood vessel, conventional blood clot diversion devices must be removed from the multifurcated zone in which they were positioned, thereby complicating blood vessel access, and increasing mortality risks to patients undergoing medical procedures. As an example, after positioning within a multifurcated zone to prevent embolic material from entering an ICA, such devices do not allow for re-access and re-crossing such that they do not allow a medical instrument access to the ICA and subsequent intracranial blood vessels connected thereto to treat intracranial conditions and emergencies.

Further, filtering devices have been introduced for use during medical procedures having a high risk of embolism, such as major cardiovascular surgery or cardiac valve repair. However, these devices have deficiencies including providing an insufficient area of protection for a particular blood vessel for which embolic material diversion is intended, occluding as a consequence of a higher embolic material burden, and failing to properly anchor to inner walls of blood vessels.

Finally, while conventional blood clot diversion devices have been contemplated for placement within the anterior circulatory system between the CCA and ECA to deflect and divert embolic material from entering the ICA, no devices have been designed for use within the posterior circulatory system to deflect and divert fine embolic material from entering into a patient's brain stem and subsequent intracranial blood vessels connected thereto.

Accordingly, needs exist for an improved embolic material diversion device that can filter and divert fine embolic material away from a particular blood vessel, while allowing for re-access and re-crossing to that blood vessel, for example, in case of a medical procedure. Further, needs exist for an improved embolic material diversion device that can expand to comply with inner walls of blood vessels of both the anterior and posterior circulatory systems reaching the brain.

### SUMMARY

In various embodiments, the present disclosure relates to an improved implantable embolic material diversion device featuring a number of openings sized to deflect and divert fine embolic material (e.g., blood clots, air bubbles, and fat globules) away from a particular branch blood vessel in a multifurcated (e.g., bifurcated) zone. The implantable embolic material diversion device may be delivered and positioned proximal to the multifurcated zone to prevent fine embolic material from entering into an inlet of a first branch blood vessel formed in the multifurcated zone and subsequent blood vessels (e.g., intracranial blood vessels) coupled to that first branch blood vessel. The embolic material diversion device may be positioned to extend between a main blood vessel and a second branch blood vessel to prevent fine embolic material from entering into the inlet of the first branch blood vessel. As an example, such positioning of the device embolic material diversion device can redirect the fine embolic material away from the intracranial circulation. In some cases, the main blood vessel and second branch blood vessel can be different portions of the same blood vessel, such as proximal and distal portions of a subclavian artery on different sides of an inlet to the vertebral artery.

In comparison to conventional devices, the embolic material diversion device provides a specific improvement of enabling re-access and re-crossing to the first branch blood vessel after positioning of the implantable embolic material diversion device within the multifurcated zone, while filtering embolic material having greater than a particular (e.g., fine) size as described herein. To enable re-access and re-crossing, the embolic material diversion device can be formed entirely from a tubular mesh body having only a single layer of a number of braided wires that form a number of openings. Such openings can have an open cell and semi-compliant (e.g., partially flexible) structure, thereby allowing for expansion from an initial size to accommodate a medical instrument and compression to the initial size after removal of the medical instrument without damage (e.g., permanent deformation) to the openings. Further, the single layer construction for the tubular mesh body is less thrombogenic than a tubular mesh body having multiple layers. As an example, a tubular mesh body having a single layer construction is less likely form a blood clot when in contact with blood and positioned in a patient's body than a different tubular mesh body having a multi-layer construction.

In some variations, the tubular mesh body can include a proximal end and a distal end, while defining a lumen between the proximal and distal ends. The tubular mesh body can function to deflect and divert embolic material within the lumen to flow from the main blood into the second branch blood vessel, with each opening sized to allow a passage therethrough of blood and to prevent the passage therethrough of fine embolic material. Each of the wires forming the tubular mesh body can be formed from a shape memory material, thereby allowing for elastic deformation of the tubular mesh body. As an example, a particular opening of the tubular mesh body can be expanded and a portion of the tubular mesh body adjacent to the particular opening can elastically deform to accommodate a medical instrument (e.g., catheter, wire, balloon, etc.) advanced to extend through the particular opening to access and extend into the first branch blood vessel. Importantly, in some cases, the embolic material diversion device is adapted for placement in blood vessels of both the anterior and posterior circulatory systems that supply blood to the brain. As an example, the embolic material diversion device may be positioned between a proximal portion of a subclavian artery or a brachiocephalic trunk and a distal portion of the subclavian artery to deflect and divert embolic material from entering the vertebral artery to prevent such embolic material from causing occlusion of the vertebral artery or a subsequent intracranial blood vessel connected thereto. As another example, the embolic material diversion device may be positioned between CCA and ECA to deflect and divert embolic material from entering the ICA to prevent such embolic material from causing occlusion of the ICA or a subsequent intracranial blood vessel connected thereto. Known delivery techniques, such as, for example, pusher wire and guiding catheter based delivery techniques may be used to advance the embolic material diversion device in proximity of and/or to the multifurcated zone.

In general, in one aspect, embodiments of the invention feature implantable embolic material diversion device. The implantable embolic material diversion device may include a tubular mesh body having a proximal end and a distal end and defining a lumen between the proximal and distal ends. The tubular mesh body may have only a single layer of a plurality of braided wires that form a plurality of openings, each braided wire having a shape memory material. Each opening may be sized to allow a passage therethrough of blood and to prevent the passage therethrough of embolic material having, in every direction of view thereof, at least one straight-line distance between two points on a perimeter of at least one cross-sectional surface in excess of 350 μm.

In various embodiments, each opening may be sized to prevent the passage therethrough of embolic material having, in every direction of view thereof, at least one straight-line distance between two points on a perimeter of at least one cross-sectional surface in excess of 200 micrometers (μm). An inscribed circle of each opening may have a diameter in a range from 180 μm to 400 μm. Each opening may have an open cell. Each opening may have a polygonal opening. In some variations, a cross-section of each of the plurality of wires can have a diameter in a range from 80 μm to 120 μm. The plurality of wires can number between 24 and 48 wires. The shape memory material can be a material selected from the group consisting of nitinol, copper-based alloy, and iron-based alloy. The shape memory material can include (e.g., be) a biocompatible material.

In various embodiments, the tubular mesh body can be self-expandable from a radially-compressed position to a radially-expanded position. In some variations, the radially-compressed position defines a delivery state of the tubular mesh body. In some variations, the radially-expanded position defines an implanted state of the tubular mesh body. The device can further include a proximal anchor at the proximal end of the tubular mesh body and a distal anchor at the distal end of the tubular mesh body. In some variations, the proximal and distal anchors form a hybrid anchoring structure. The proximal anchor can include a looped anchor structure formed from a subset of the plurality of wires. The distal anchor can include a crowned anchor structure formed from a subset of the plurality of wires.

In general, in another aspect, embodiments of the invention feature a method of delivering an implantable embolic material diversion device to a multifurcated zone dividing a main blood vessel into at least two branch blood vessels. The method can include advancing the implantable embolic material diversion device, via a delivery device, in proximity of the multifurcated zone. The implantable embolic material diversion device may include a tubular mesh body having a proximal end and a distal end and defining a lumen between the proximal and distal ends. The tubular mesh body may have only a single layer of a plurality of braided wires that form a plurality of openings, each braided wire having a shape memory material. Each opening may be sized to allow a passage therethrough of blood and to prevent the passage therethrough of embolic material having, in every direction of view thereof, at least one straight-line distance between two points on a perimeter of at least one cross-sectional surface in excess of 350 μm. The method can include releasing the implantable embolic material diversion device from the delivery device into the multifurcated zone.

In various embodiments, the delivery device can include a pusher wire and a guiding catheter having a diameter in a range from 5 French (Fr) (1.67 mm) to 7 Fr (2.33 mm). In some cases, the delivery device can further include a guiding wire. In some variations, releasing the implantable embolic material diversion device from the delivery device (e.g., guiding catheter) into the multifurcated zone (e.g., using the pusher wire) causes the tubular mesh body to expand from a radially-compressed position to a radially-expanded position. The method can include at least partially retracting the implantable embolic material diversion device into the delivery device from the multifurcated zone, thereby causing at least part of the tubular mesh body to compress into the radially-compressed position. The implantable embolic material diversion device can further include a proximal anchor at the proximal end of the tubular mesh body and a distal anchor at the distal end of the tubular mesh body. In some variations, releasing the implantable embolic material diversion device from the delivery device into the multifurcated zone causes (i) the proximal anchor to anchor at least the proximal end of the tubular mesh body to a wall of the main blood vessel on a proximal side of an inlet to a first branch blood vessel and (ii) the distal anchor to anchor at least the distal end of the tubular mesh body to a wall of a second branch blood vessel on a distal side of the inlet to the first branch blood vessel. The main blood vessel can include a proximal portion of a subclavian artery or a brachiocephalic trunk, the first branch blood vessel can include a vertebral artery, and the second branch blood vessel can include a distal portion of the subclavian artery. The main blood vessel can include a common carotid artery, the first branch blood vessel can include an internal carotid artery, and the second branch blood vessel can include an external carotid artery.

In various embodiments, the method can further include advancing a medical instrument through a particular one of the openings and into an inlet to a first branch blood vessel, thereby causing (i) expansion of the particular opening from an initial size and (ii) elastic deformation of a portion of the tubular mesh body adjacent to the particular opening. In some variations, the method can further include retracting the medical instrument from the particular opening and out of the inlet to the first branch blood vessel, whereby the particular opening returns to its initial size when the medical instrument is retracted from the particular opening.

These and other objects, along with advantages and features of the embodiments of the present invention herein disclosed, will become more apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A is a schematic side perspective view of an implantable embolic material diversion device having a tubular mesh body, in accordance with some embodiments;

FIG. 1B is a schematic side perspective view of an implantable embolic material diversion device having a tubular mesh body, in accordance with some embodiments;

FIG. 1C is a schematic side perspective view of an implantable embolic material diversion device having a tubular mesh body, in accordance with some embodiments;

FIG. 1D is a schematic front view of an implantable embolic material diversion device having a tubular mesh body, in accordance with some embodiments;

FIG. 2A is a segment of schematic side view of the tubular mesh body of FIG. 1C, in accordance with some embodiments;

FIG. 2B is a segment of schematic side view of the tubular mesh body of FIG. 1C, in accordance with some embodiments;

FIG. 2C is a cross-sectional view taken along lines 210-210 of the tubular mesh body of FIG. 2A, in accordance with some embodiments;

FIG. 3A is a side cross-sectional view of a multifurcated zone of a patient's body including an implantable embolic material diversion device, in accordance with some embodiments;

FIG. 3B is a side cross-sectional view of a multifurcated zone of a patient's body including an implantable embolic material diversion device, in accordance with some embodiments;

FIG. 4 shows a cross-sectional surface of a blood clot included in embolic material is shown, in accordance with some embodiments;

FIG. 5A shows a photographic image of a tubular mesh body of the implantable embolic material diversion device, in accordance with some embodiments; and

FIG. 5B shows a photographic image of a tubular mesh body of the implantable embolic material diversion device, in accordance with some embodiments; and

FIG. 6 shows a photographic image of a medical instrument extending through an opening of the tubular mesh body of the implant, in accordance with some embodiments.

### DETAILED DESCRIPTION

Embodiments of the present disclosure are directed to a novel design and construction for an implantable embolic material diversion device that is functionally superior to conventional embolic material diversion devices by deflecting and diverting fine embolic away from entering a particular branch blood vessel, while enabling access (e.g., via a medical instrument) to the branch blood vessel and other blood vessels following the branch blood vessel via the device's open cell structure, semi-compliant (e.g., partially flexible) structure, and shape memory properties. As an example, the implantable embolic material diversion device can deflect and divert fine embolic away from entering a particular branch blood vessel of either of the anterior and posterior circulatory systems that supply blood to the brain. Further, a single-layer wire mesh construction of the implantable embolic material diversion device provides reduced thrombogenicity when positioned in a patient's body relative to a multi-layered wire mesh construction, while filtering fine embolic having greater than a particular size as described herein.

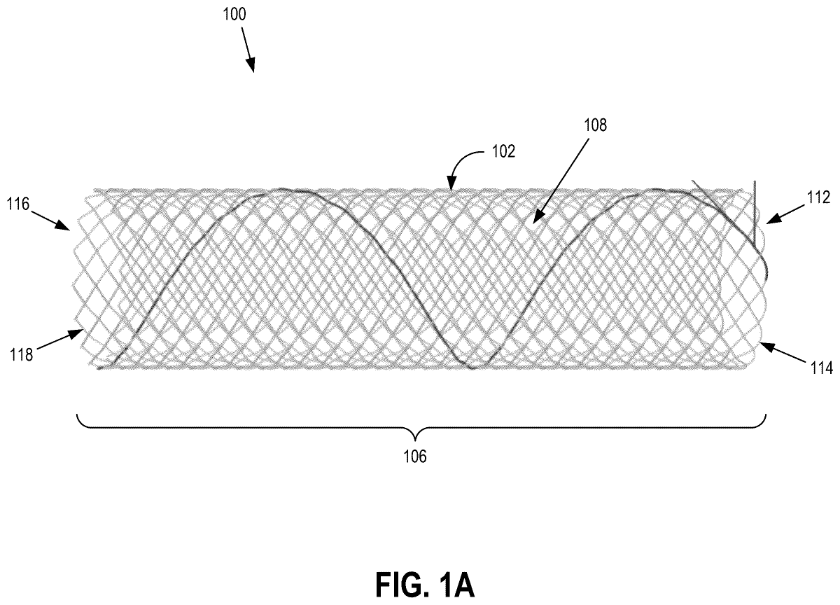

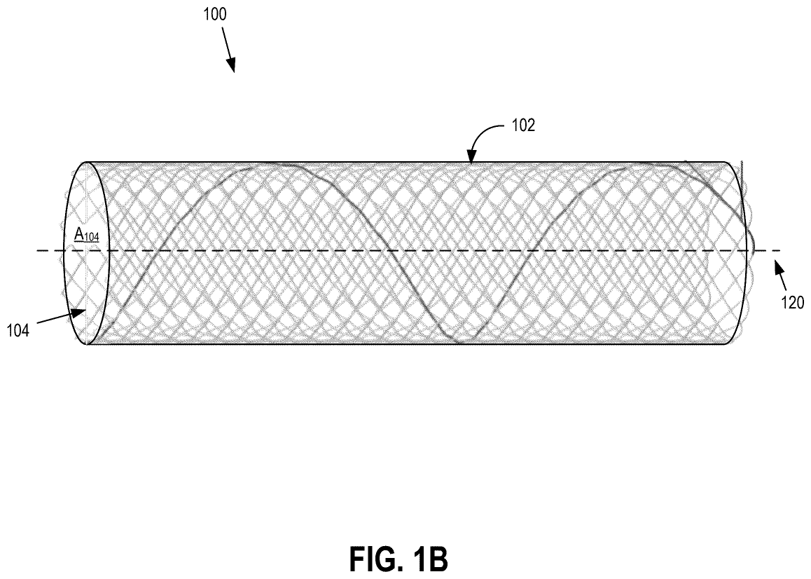

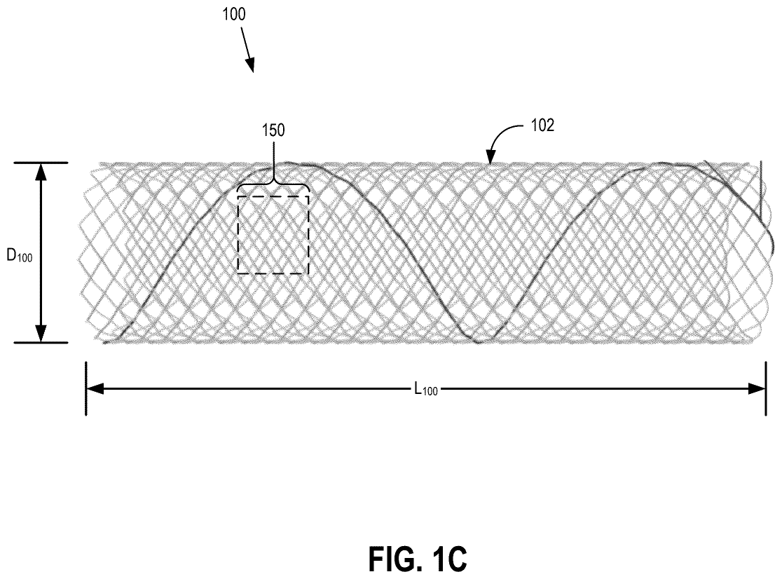

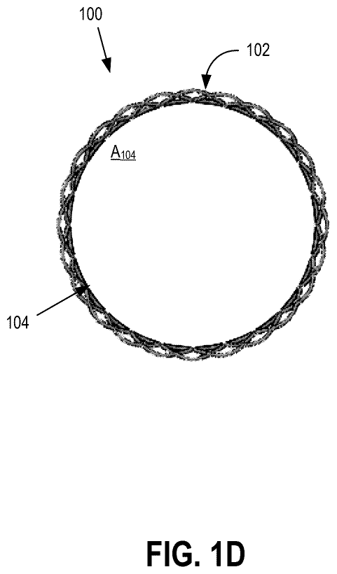

Referring to FIGS. 1A-1D, in some embodiments, the implantable embolic material diversion device 100 (also referred to as “implant 100”) includes tubular mesh body 102 having a proximal end 112 and a distal end 116. The tubular mesh body 102 can define a lumen 104 (e.g., as shown in FIG. 1D) extending between the proximal end 112 and the distal end 116 of the tubular mesh body 102. In some cases, the implant 100 is formed entirely from the tubular mesh body 102.

In some embodiments, the tubular mesh body 102 has only a single layer of a number of braided wires 106 that form a number of openings 108, such that the tubular mesh body 102 is formed entirely of the single layer of the wires 106. In some cases, as an example, the braiding of the wires 106 may form a lattice structure defining the openings 108 of the tubular mesh body 102. As described herein, the single layer construction of the tubular mesh body 102 from the wires 106 provides the specific advantage of a reduction in thrombogenicity of the implant 100 relative to devices including more than one layer of wire mesh or other structures combined with a single layer of wire mesh. In some cases, the tubular mesh body 102 may be formed as a self-expandable stent.

In some embodiments, each opening 108 of the tubular mesh body 102 can be sized to allow a passage (e.g., flow) therethrough of blood and to prevent the passage (e.g., flow) therethrough of embolic material. Some non-limiting examples of embolic material can include blood clots, air bubbles, and fat globules. In some cases, the embolic material can include thromboembolic material derived from a thrombus. Thus, the openings 108 of the tubular mesh body 102 can be sized to cause no change or an insignificant change to blood flow to a blood vessel for which the implant 100 diverts embolic material, thereby allowing at least red blood cells and plasma to flow therethrough. In some cases, the openings 108 of the tubular mesh body 102 can be sized to cause no change or an insignificant change to cerebral blood flow when the tubular mesh body is positioned to divert embolic material from entering an ICA or vertebral artery. As an example, when the implant 100 is positioned to deflect and divert particularly sized embolic material away from entering an inlet to an ICA or a vertebral artery, the openings 108 of the tubular mesh body 102 can be sized to cause less than a 5% reduction in cerebral blood flow relative to cerebral blood flow prior to positioning of the implant 100 to deflect and divert particularly sized embolic material away from entering the inlet to the ICA or vertebral artery. As another example, when the implant 100 is positioned to deflect and divert particularly sized embolic material away from entering an inlet to an ICA or a vertebral artery, the openings 108 of the tubular mesh body 102 can be sized such that cerebral blood flow is greater than 30 milliliters (mL) per 100 grams of brain tissue per minute.

In some embodiments, embolic material prevented from passing through the openings 108 of the tubular mesh body 102 may be defined as embolic material having, in every direction of view thereof, at least one straight-line distance between two points on a perimeter of at least one cross-sectional surface (e.g., of all cross-sectional surfaces for that view) in excess of a straight-line distance in a range of 200 micrometers (μm) to 350 μm. In some cases, embolic material prevented from passing through the openings 108 of the tubular mesh body 102 may be defined as embolic material having, in every direction of view thereof, at least one straight-line distance between two points on a perimeter of at least one cross-sectional surface in excess of 350 micrometers (μm). In some cases, embolic material prevented from passing through the openings 108 of the tubular mesh body 102 may be defined as embolic material having, in every direction of view thereof, at least one straight-line distance between two points on a perimeter of at least one cross-sectional surface in excess of 200 μm. Thus, the openings 108 formed by the wires 106 may be sized as desired based on the braiding of the wires 106, a size (e.g., cross-sectional area) of each of the wires 106, and/or the number of wires 106 included in the tubular mesh body 102 to prevent passage therethrough of embolic material (e.g., into an inlet of a particular branch blood vessel and subsequent blood vessels connected thereto) having greater than a particular size, while allowing passage therethrough of blood.

In some embodiments, one or more (e.g., each) of the wires 106 that are braided to form the tubular mesh body 102 may have (e.g., be formed from) a shape memory material. A shape memory material may be a material with natural shape memory characteristics, such that it is elastically deformable and/or biased toward a particular position. Some non-limiting examples of the shape memory material include nitinol (nickel-titanium alloy), copper-based alloy, and iron-based alloy. In some cases, one or more (e.g., each) of the wires 106 that are braided to form the tubular mesh body 102 may be made (e.g., entirely) of a shape memory material selected from the group consisting of nitinol, copper-based alloy, and iron-based alloy. In some cases, one or more of the wires 106 that are braided to form the tubular mesh body 102 may have (e.g., be formed from) a non-shape memory material. A non-limiting examples of the non-shape memory material includes a cobalt and chromium (chrome-cobalt) alloy, platinum, and fiber-glass. In some cases, one or more of the wires 106 that are braided to form the tubular mesh body 102 may be made entirely of a non-shape memory material. When at least some of (e.g., all of) the wires 106 have (e.g., are formed from) a shape memory material, the tubular mesh body 102 can have shape memory characteristics. As an example, shape memory characteristics of the tubular mesh body 102 can allow for elastic deformation of the tubular mesh body 102 and/or biasing of the tubular toward a particular position, such biasing toward a fully radially-expanded position as described herein. Based on at least some of (e.g., all of) the wires 106 having a shape memory material and (e.g., each of) the openings 108 having an open cell, the tubular mesh body 102 can be semi-compliant, such that the tubular mesh body is partially flexible and elastically deformable. Thus, the tubular mesh body 102 may expand to and comply with the dimensions of inner wall(s) of particular blood vessels.

In some embodiments, each of the shape memory material and the non-shape memory material may be a biocompatible material that is non-toxic, non-irritating, and safe to use in contact with the body (e.g., interior of a body, such as a blood vessel). In some cases, the shape memory material and non-shape memory material may be radiopaque, such that they are visible under ultrasound, X-rays, and Magnetic Resonance Imaging (MRI). In some cases, one or more the wires 106 that are braided to form the tubular mesh body 102 may have a coating configured to reduce thrombogenicity of the wires 106 having the coating relative the wires 106 lacking the coating. As an example, one or more (e.g., each) of the wires 106 that are braided to form the tubular mesh body 102 may be coated on an outer surface with a material configure to reduce thrombogenicity of the wires 106, such as heparin. In some cases, the wires 106 may number between, for example, 24 and 64 wires, with the tubular mesh body 102 being formed entirely from such a number of wires 106. A cross-section (e.g., cross-sectional area) of each of the wires 106 can, for example, have a diameter in a range from 80 μm to 120 μm. In some cases, the cross-section (e.g., cross-sectional area) of the wires 106 may vary, such that a first wire 106 has a different cross-section (e.g., cross-sectional area) than a second wire 106. A thickness of each of the wires 106 can, for example, be in a range from 80 μm to 120 μm. A particular wire 106 of the wires 106 can have a variable thickness or uniform thickness.

In some embodiments, the tubular mesh body 102 is expandable between a radially-compressed position and a radially-expanded position. The tubular mesh body 102 may be expandable between a fully radially-compressed position and a fully radially-expanded position, with the radially-compressed position and the radially-expanded position being in the range of the fully radially-compressed position and a fully radially-expanded position. As shown in FIGS. 1A-1D, the tubular mesh body 102 has the fully radially-expanded position in which no constraining forces are acting to compress the tubular mesh body 102. In some cases, based on at least some of the wires 106 having a shape memory material and shape memory characteristics derived therefrom, the tubular mesh body 102 can be self-expandable from the radially-compressed position to the radially-expanded position. The radially-compressed position may define a delivery state of the tubular mesh body 102 in which the implant 100 and tubular mesh body 102 are compressed within a delivery device. At the delivery state, the implant 100 can be caused to enter a patient's body and advanced to a particular multifurcated zone in the patient's body. The radially-expanded position may define an implanted state of the tubular mesh body 102 in which the implant 100 and its tubular mesh body 102 are expanded up to and compliant with the dimensions of inner wall(s) of the blood vessel(s) in which the implant 100 is positioned. At the implanted state, the implant 100 can be positioned within a particular multifurcated zone in the patient's body, such that the implant 100 can deflect and divert embolic material originating from a main blood vessel away from a first branch blood vessel toward a second branch blood vessel, thereby preventing the embolic material from entering an inlet to first branch blood vessel (e.g., and subsequent blood vessels connected thereto), allowing blood to continue to flow into the inlet to the first branch blood vessel, and causing the embolic material to flow into and through the second branch blood vessel. Further, at the implanted state, the implant 100 can be positioned and have openings 108 sized such that the implant 100 causes little to no change in blood flow to the first branch blood vessel relative to blood flow into the first branch blood vessel before positioning of the implant 100. Thus, at the implanted state and when positioned within a multifurcated zone, at least a portion (e.g., wall) of the tubular mesh body 102 can cover an inlet to the first branch blood vessel, thereby preventing embolic material having greater than a particular size from entering the first branch blood vessel and diverting such embolic material toward and into the second branch blood vessel.

As shown in FIG. 1B, in some cases, the tubular mesh body 102 can be self-expandable about an axis 120 (e.g., central axis) extending through (e.g., a center of) the lumen 104 and through the proximal and distal ends 112, 116. When the tubular mesh body 102 has the fully radially-expanded position, the axis 120 may be linear. When the tubular mesh body 102 has any position other than the fully radially-expanded position, the axis 120 may be linear or non-linear based on constraining forces acting on the tubular mesh body 102 to compress at least a portion of the tubular mesh body 102. The tubular mesh body 102 can have a variable diameter D₁₀₀ and a variable length L₁₀₀ determined by a position of the tubular mesh body 102 or portion(s) thereof between the fully radially-compressed position and the fully radially-expanded position, with the diameter D₁₀₀ being able to vary along the length L₁₀₀. As an example, the tubular mesh body 102 can have an increased diameter D₁₀₀ and a reduced length L₁₀₀ when expanded to the radially-expanded position relative to the radially-compressed position. Because the diameter D₁₀₀ can vary along the length L₁₀₀, a cross-sectional area A₁₀₄ of the lumen 104 defined by the tubular mesh body 102 can also vary along the length L₁₀₀.

In some embodiments, a fully radially expanded position of the tubular mesh body 102 may be a nominal position at which the tubular mesh body 102 is manufactured. At the nominal position, the tubular mesh body 102 may have a (e.g., outer diameter) diameter D₁₀₀ in a range from 5 millimeters (mm) to 12 mm and a length L₁₀₀ in a range from 30 mm to 60 mm. In some cases, as an example for anterior circulation (e.g., placement in a multifurcated zone for a diverting embolic material from an ICA), the tubular mesh body 102 may have a diameter D₁₀₀ in a range from 5 mm to 8 mm and a length L₁₀₀ in a range from 40 mm to 60 mm. In some cases, as an example for posterior circulation (e.g., placement in a multifurcated zone for a diverting embolic material from a vertebral artery), the tubular mesh body 102 may have a diameter D₁₀₀ in a range from 9 mm to 12 mm and a length L₁₀₀ in a range from 30 mm to 50 mm. A cross-sectional area A₁₀₄ of the lumen 104 may vary between, for example, 19.63 mm² and 113.1 mm² based on the diameter D₁₀₀ of the tubular mesh body 102.

In some embodiments, application of constraining forces to the tubular mesh body 102 sufficient to overcome shape memory (e.g., tensile and/or elastic) characteristics of at least some of the wires 106 can cause at least a portion (e.g., segment) of the tubular mesh body 102 to compress from the fully radially-expanded position toward the radially-compressed position. When a constraining force is applied to the tubular mesh body 102 and compresses at least a portion of the tubular mesh body 102, the length L₁₀₀ of the compressed portion of the tubular mesh body 102 can increase and both the diameter D₁₀₀ of the compressed portion of the tubular mesh body 102 and the cross-sectional area A₁₀₄ of the lumen 104 can decrease. As an example, a delivery device for the implant 100 can apply a constraining force to the tubular mesh body 102, thereby compressing the tubular mesh body 102 or a portion thereof to the radially-compressed position. Upon removal of the constraining force from the tubular mesh body 102, the tubular mesh body 102 can expand toward the radially-expanded position that can be less than or equivalent to the fully radially-expanded position (e.g., due to the shape memory material of the wires 106). In use and when positioned in a multifurcated zone, the tubular mesh body 102 can expand toward the fully radially-expanded position and up to the particular radially-expanded position allowed by the dimensions of the inner walls of the blood vessels (e.g., main blood vessel and second branch blood vessel) in the area of the multifurcated zone in which the tubular mesh body 102 is positioned, thereby complying with the dimensions of the inner walls of the blood vessels.

In some embodiments, the implant 100 can include a proximal anchor 114 at the proximal end 112 of the tubular mesh body 102. The proximal anchor 114 may be disposed and/or formed directly on the proximal end 112. In some cases, the implant 100 can include a distal anchor 118 at the distal end 116 of the tubular mesh body 102. The distal anchor 118 may be disposed and/or formed directly on the distal end 116. Together, the proximal anchor 114 and the distal anchor 118 can form a hybrid anchoring structure for the implant 100 when the proximal and distal anchors 114, 118 have different structures. In some cases, as shown in FIGS. 1A-1D, the proximal anchor 114 can be a looped anchor structure formed from a subset of the wires 106. The looped anchor structure may define the proximal end 112 of the tubular mesh body 102 and include a number of wire loops. The wire loops formed from the wires 106 may be closed such that ends of the wires 106 are not exposed at the looped anchor structure. In some cases, the looped anchor structure can provide improved re-crossing and re-access abilities for the tubular mesh body 102 relative to a crowned anchor structure. In some cases, a particular wire loop may be curved or angular. An angular wire loop may form an angle in the range of, for example 1 degree (°) to 179°. In some cases, as shown in FIGS. 1A-1D, the distal anchor 118 can be a crowned anchor structure formed from a subset of the wires 106. The crowned anchor structure may define the distal end 116 of the tubular mesh body 102 and include a number of points. The points may be formed from the open ends of the wires 106 such that the ends of the wires 106 are exposed at the crowned anchor structure. In some cases, the crowned anchor structure can provide increased stability and anchoring of the tubular mesh body 102 to an inner wall of a blood vessel relative to the looped anchor structure. In some cases, the proximal and distal anchors 114, 118 may have alternative structures (e.g., different from the looped and crowned anchor structures). As an example, in some cases, the proximal anchor 114 can be a crowned anchor structure formed from a subset of the wires 106 and the distal anchor 118 can be a looped anchor structure.

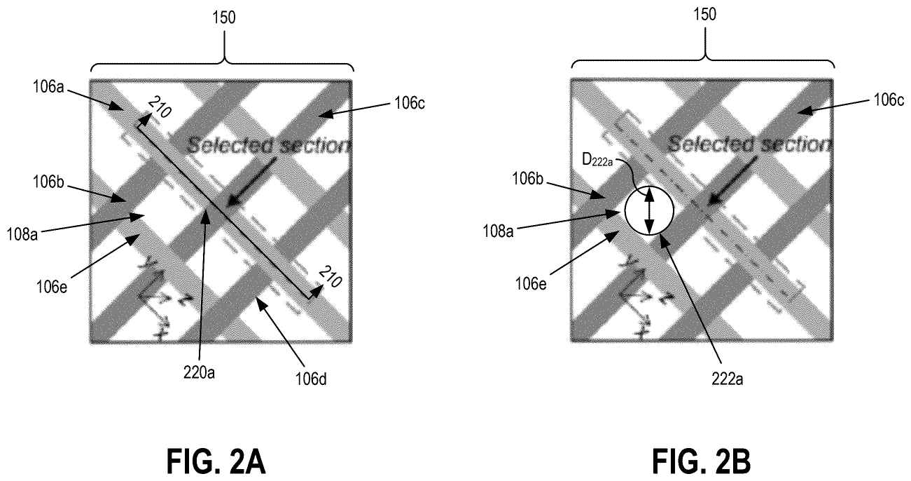

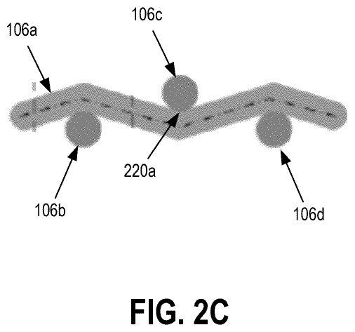

Referring to FIGS. 2A and 2B, a segment 150 of the tubular mesh body 102 viewed normal to the surface of the tubular mesh body 102 is shown. The segment 150 of the tubular mesh body 102 can include a number of braided wires 106 forming a number of openings 108, including the first, second, third, and fourth wires 106a-d. Each opening 108 can be formed by braiding of a subset of the wires 106, with each opening 108 formed from segments of a subset of wires 106. In some cases, the openings 108 can have a polygonal shape defined by segments of the wires 106. As shown for example in FIGS. 2A and 2B, the polygonal shape may be a quadrangle, such as a parallelogram. Parallelograms include squares, rectangles, and lozenges.

Referring to FIGS. 2A and 2C, each opening 108 can be formed by a number of joints 220. At each joint 220 forming a portion of a particular opening 108, at least two wires 106 are adjacent and overlapping. In some cases, at each joint 220, at least two wires 106 are adjacent, overlapping, and in contact with each other. Each opening 108 of the tubular mesh body 102 can have an open cell, such that each joint 220 forming that opening 108 is unconstrained. A joint 220 can be referred to as unconstrained when each wire 106 forming that joint 220 is not coupled (e.g., fastened) to an adjacent wire 106 forming that joint 220. When an opening 108 has an open cell, each joint 220 forming that opening 108 may be moveable and unconstrained (e.g., by any fastener). Referring to FIG. 2C, a cross-sectional view taken along lines 210-210 of the segment 150 of the tubular mesh body 102 of FIG. 2A is shown. The wire 106a can positioned over the wires 106b and 106d and under the wire 106c. As shown in FIGS. 2A and 2C, the overlapping and adjacent positioning of the wires 106a and 106c forms the joint 220a of the opening 108a, with the wire 106c crossing over the wire 106a to form the joint 220a. The joint 220a is unconstrained due to the wires 106a, 106c not being coupled together, such that the wires 106a, 106c can move and change a position of the joint 220a along the tubular mesh body 102. From the open cell structures of the openings 108, the openings 108 can be semi-compliant, such that the openings are at least partially flexible. For example, an opening 108 may be semi-compliant to enable re-access and re-crossing as described herein, such that the opening can expand to accommodate a medical instrument. the openings 108 may be semi-compliant based on the adjacent (e.g., overlapping) positioning of wires 106 that form the tubular mesh body 102.

In some embodiments, referring to FIG. 2B, each opening 108 can have an inscribed circle 222. An inscribed circle 222 of an opening 108 refers to the largest circle that can be drawn inside the opening 108 and tangent to at least three of the sides (e.g., segments of the subset of wires 106) forming the opening 108. An inscribed circle 222 has a diameter D₂₂₂. As shown in FIG. 2B, the opening 108a can have an inscribed circle 222a that is tangent to at least three sides of the opening 108a defined by the wires 106b, 106c, and 106e forming the opening 108a, with the inscribed circle 222a having a diameter D₂₂₂ₐ. In some cases, the inscribed circle 222 of each opening 108 has a diameter D₂₂₂ in a range from 180 μm to 400 μm. Such a diameter D₂₂₂ for the inscribed circle 222 can enable each opening 108 to prevent passage therethrough of as embolic material having, in every direction of view thereof, at least one straight-line distance between two points on a perimeter of at least one cross-sectional surface in excess of 350 μm (e.g., in excess of 200 μm). The inscribed circle 222 of each opening 108 can be sized to prevent passage therethrough of particular embolic material as described herein.

In some embodiments, based on the self-expandable properties of the tubular mesh body 102 of the implant 100 and, sizes (e.g., diameters D₂₂₂ for the inscribed circles 222) of the openings 108 can be determined by a position of the tubular mesh body 102 or portion(s) thereof between the radially-compressed position and the radially-expanded position. Accordingly, the sizes of the openings 108 can vary based on a position of the tubular mesh body 102 or portion(s) thereof between the fully radially-compressed position and the fully radially-expanded position. As an example, when tubular mesh body 102 is in the radially-compressed position, the spacing between and sizes of the openings 108 may be reduced. As another example, when tubular mesh body 102 is in the radially-expanded position, spacing between and sizes of the openings 108 may increase relative to the radially-compressed position (e.g., through the action of the shape memory material). Sizes of different openings 108 may vary differently based on their position along the length L₁₀₀ of the tubular mesh body 102. As an example, a first opening 108 positioned proximal the proximal end 112 and distal the distal end 116 may have a size (e.g., diameter D₂₂₂ of its inscribed circle 222) greater than, less than, or the same as a different, second opening 108 positioned distal the proximal end 112 and proximal the distal end 116 based on a position of the tubular mesh body 102 or portion(s) thereof between the fully radially-compressed position and the fully radially-expanded position. Further, in some cases, a particular opening 108 can change (e.g., expand) from an initial size in response to a medical instrument (e.g., catheter, wire, balloon, etc.) entering and extending through the opening 108 and pushing against the wires 106 forming the opening 108. Upon removal of the medical instrument from the particular opening 108, that opening 108 can revert (e.g., compress) back to its initial size from before the medical instrument entered and extended through the opening 108 (e.g., due to the shape memory material of the wires 106). In some cases, a medical instrument entering and extending through the opening 108 and pushing against the wires 106 forming the opening 108 can cause elastic deformation of the portion of the tubular mesh body 102 adjacent to (e.g., surrounding) the particular opening 108, thereby causing sizes of openings 108 included in that portion to compress from their initial sizes. Upon removal of the medical instrument from the particular opening 108, the adjacent openings 108 included in that portion of the tubular mesh body 102 can expand back to their initial sizes from before the medical instrument entered and extended through the opening 108 (e.g., due to the shape memory material of the wires 106).

The particular opening 108 can revert back to its initial size from before the medical instrument entered and extended through the opening 108 without damage (e.g., permanent deformation) to the particular opening 108, adjacent openings 108, wires 106 forming the opening 108 and adjacent openings 108, and the tubular mesh body 102. Such properties enable re-crossing and re-access of a blood vessel for which embolic material is diverted, such that the medical instrument can extend into the blood vessel and subsequent blood vessels connected thereto while the implant remains positioned within the multifurcated zone. In some cases, the openings 108 having open cells can enable the ability for the sizes of the openings 108 to vary, as adjacent wires 106 can change relative position and joints 220 forming each of the openings can shift along segments of the wires 106 forming each of the openings 108.

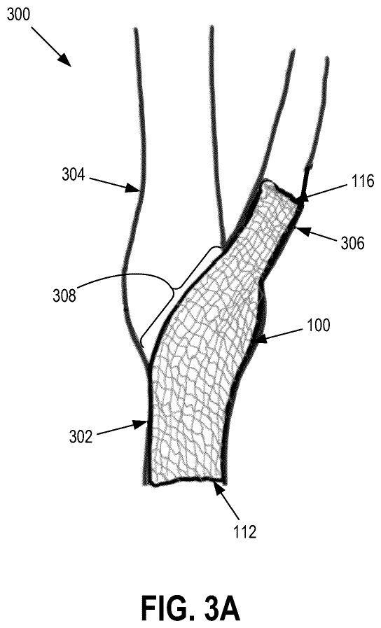

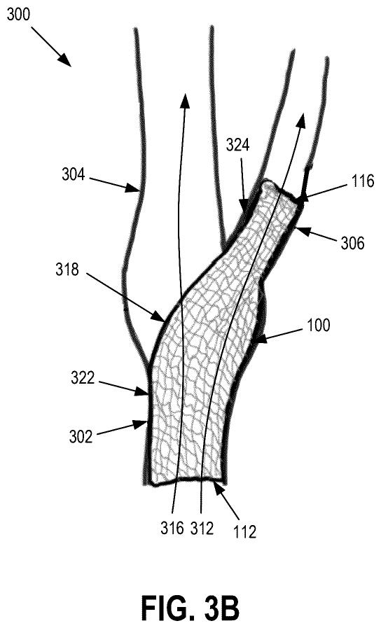

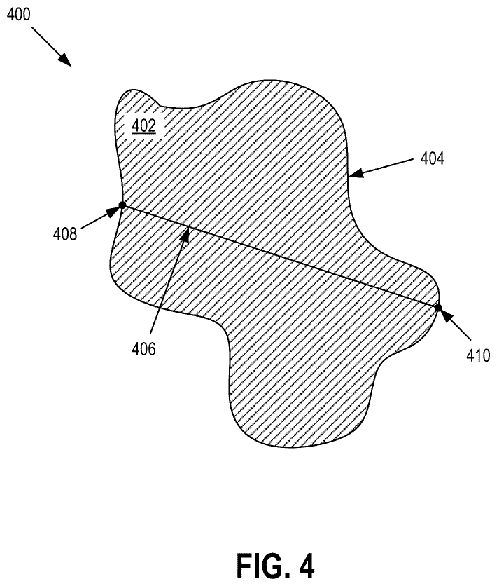

Referring to FIGS. 3A and 3B, a side cross-sectional view of a multifurcated zone 300 of a patient's body including an implant 100 positioned therein is shown. The multifurcated zone 300 can be at least part of a multifurcated blood vessel including a main blood vessel 302 at least bifurcating into at least two branch blood vessels. The at least two branch blood vessels may include a first branch blood vessel 304 and a second branch blood vessel 306. Blood in the circulatory system flows from the main blood vessel 302 into each of the first and second branch blood vessels 304, 306. In the context of the implant 100, the multifurcated blood vessel may be an anterior or posterior circulatory system supplying blood to the brain, such that the implant 100 can be positioned within either of the anterior or posterior circulatory system supplying blood to the brain to prevent ischemic stroke. For an anterior circulatory system use case, the main blood vessel 302 may be the CCA, the first branch blood vessel 304 may be an ICA, and the second branch blood vessel 306 may be an ECA. For a posterior circulatory system use case, the main blood vessel 302 may be a proximal portion (e.g., end) of a subclavian artery or a brachiocephalic trunk (e.g., depending on a patient's anatomy), the first branch blood vessel 304 may be a vertebral artery, and the second branch blood vessel 306 may be a distal portion (e.g., end) of the subclavian artery. Thus, for both the anterior and posterior circulatory system use cases, the implant 100 can function to prevent embolic material (e.g., blood clots) having greater than a particular size from flowing into and potentially occluding intracranial blood vessels by diverting such material away therefrom. Accordingly, the multifurcated zone 300 shown in FIGS. 3A and 3B can represent any of the posterior and anterior circulatory system use cases described herein. Further, the multifurcated zone 300 may be any applicable multifurcated zone within a patient's body.

In some embodiments, as shown for example in FIGS. 3A and 3B, the implant 100 may be delivered to and positioned within the multifurcated zone 300. Based on the shape memory properties of the tubular mesh body 102, the implant 100 can self-expand to the radially-expanded position to conform to and comply with the dimensions of the inner walls of the blood vessels of the multifurcated zone 300. The implant 100 can be positioned so that the proximal anchor 114 anchors at least the proximal end 112 of the tubular mesh body 102 to an inner wall of the main blood vessel 302. The proximal anchor 114 can anchor and retain the proximal end 112 of the tubular mesh body 102 to the inner wall of the main blood vessel 302 on a proximal side of an inlet 308 to the first branch blood vessel 304. Further, the implant 100 can be positioned so that the distal anchor 118 anchors at least the distal end 116 of the tubular mesh body 102 to an inner wall of the second branch blood vessel 306. The distal anchor 118 can anchor and retain the distal end 116 of the tubular mesh body 102 to the inner wall of the second branch blood vessel 306 on a distal side of the inlet 308 to the first branch blood vessel 304. The distal and proximal anchors 114, 118 can firmly contact the inner walls of the main blood vessel 302 and second branch blood vessel 306, respectively. Such contact can cause a growth of the inner walls of the blood vessels into the tubular mesh body 102 of the implant 100, and strongly anchors the implant 100 to the inner walls of the blood vessels, thereby preventing accidental displacement of the implant 100 within the multifurcated zone 300 after initial placement.

In some embodiments, when the implant 100 is positioned in the multifurcated zone 300 as shown in FIGS. 3A and 3B, the implant 100 can function to deflect and divert embolic material flowing from the main blood vessel 302 away from the first branch blood vessel 304 toward the second branch blood vessel 306. As an example, the implant 100 can cause particular embolic material to flow in first direction 312 from the main blood vessel 302 to the second branch blood vessel 306, while still allowing for blood to flow into the first branch blood vessel 304 for which embolic material is diverted along the second direction 316. By deflecting and diverting embolic material flowing from the main blood vessel 302 away from the first branch blood vessel 304 toward the second branch blood vessel 306, the implant 100 prevents the diverted embolic material from entering into subsequent blood vessels connected to the first branch blood vessel 304. A portion 318 of the tubular mesh body 102 positioned adjacent to the inlet 308 to the first branch blood vessel 304 can cause deflection and diversion of embolic material flowing toward the inlet 308 via the wires 106 forming that portion 318. As embolic material flows toward and contacts the portion 318, the wires 106 forming that portion 318 prevent passage of the embolic material through the openings 108 included in that portion 318, such that those wires 106 block and deflect the embolic material away from the inlet 308 toward the second branch blood vessel 306. As the embolic material is blocked and deflected from the portion 318 of the tubular mesh body 102 at the inlet 308, the embolic material is diverted and forced to flow into the second branch blood vessel 306. By diverting the embolic material from entering the first branch blood vessel 304 via the inlet 308, the implant 100 and its portion 318 thereof prevent such embolic material from occluding subsequent blood vessels (e.g., intracranial blood vessels) that come after the first branch blood vessel 304 and are connected thereto. When the first branch blood vessel 304 is an ICA or a vertebral artery leading to subsequent intracranial blood vessels of the brain, the prevention of embolic material from entering the brain can therefore reduce and eliminate risk of ischemic stroke in the patient having the multifurcated zone 300.

In some embodiments, based on the multifurcated zone 300 being, for example, a multifurcated zone in any of the anterior and posterior circulatory systems, one or more implants 100 may be positioned into one or more respective multifurcated zones 300 in a patient's body. In some cases, a system of two or more implants 100 may be positioned into two or more respective multifurcated zones 300 in a patient's body. For a multifurcated zone 300, the main blood vessel 302 may be a right CCA, the first branch blood vessel 304 may be a right ICA, and the second branch blood vessel 306 may be a right ECA. For a multifurcated zone 300, the main blood vessel 302 may be a left CCA, the first branch blood vessel 304 may be a left ICA, and the second branch blood vessel 306 may be a left ECA. For a multifurcated zone 300, the main blood vessel 302 may be a right subclavian artery or brachiocephalic trunk, the first branch blood vessel 304 may be a right vertebral artery, and the second branch blood vessel 306 may be the right subclavian artery. For a multifurcated zone 300, the main blood vessel 302 may be a left subclavian artery, the first branch blood vessel 304 may be a left vertebral artery, and the second branch blood vessel 306 may be the left subclavian artery. Thus, an implant 100 may be positioned in one or more multifurcated zones 300 of a patient's body, such as the left and right CCAs and the left and right prevertebral subclavian arteries.

Referring to FIG. 4, a cross-sectional surface 402 of a blood clot 400 (also referred to as an “embolus”) included in the embolic material is shown. The blood clot 400 can be an example of embolic material having, in every direction of view thereof, at least one straight-line distance between two points on a perimeter of at least one cross-sectional surface in excess of 350 μm (e.g., 200 μm). As shown in FIG. 4, the blood clot 400 has a cross-sectional surface 402 with a perimeter 404. In some cases, a straight line distance 406 between a first point 408 and a second point 410 on the perimeter 404 can exceed, for example, 350 μm, such that blood clot 400 has at least one straight-line distance between two points on a perimeter of at least one cross-sectional surface in excess of 350 μm. In some cases, the straight line distance 406 between the first point 408 and the second point 410 on the perimeter 404 can exceed, for example, 200 μm, such that blood clot 400 has at least one straight-line distance between two points on a perimeter of at least one cross-sectional surface in excess of 200 μm. Thus, each of the openings 108 of the tubular mesh body 102 of the implant 100 may be sized to prevent the passage therethrough of embolic material including the blood clot 400, while allowing passage of blood therethrough.

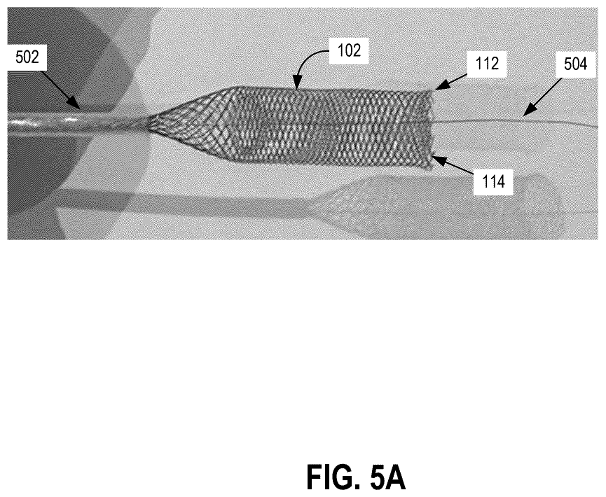

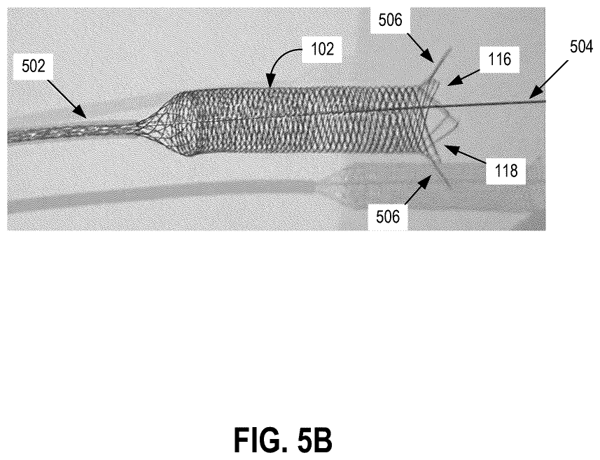

Referring to FIG. 5A, a photographic image of the proximal anchor 114 at the proximal end 112 of the tubular mesh body 102 of the implant 100 is shown. As shown in FIG. 5A, the proximal anchor 114 is a looped anchor structure formed from a subset of the wires 106 directly on the proximal end 112 of the tubular mesh body 102. The tubular mesh body 102 is shown partially retracted into a guiding catheter 502 and disposed about a guiding wire 504.

Referring to FIG. 5B, a photographic image of the distal anchor 118 at the distal end 116 of the tubular mesh body 102 of the implant 100 is shown. As shown in FIG. 5B, the distal anchor 118 is a crowned anchor looped anchor structure formed from a subset of the wires 106 directly on the distal end 116 of the tubular mesh body 102. The tubular mesh body 102 is shown partially retracted into a guiding catheter 502 and disposed about a guiding wire 504. As shown, ends 506 of a subset of the wires 106 are exposed at the crowned anchor structure.

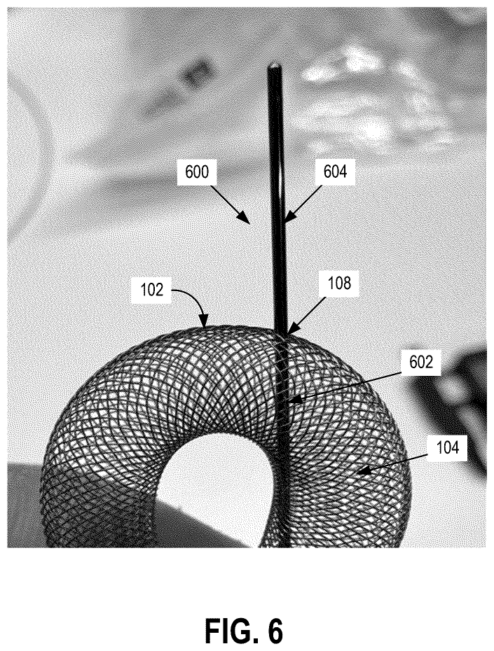

Referring to FIG. 6, a photographic image of a medical instrument 600 extending through an opening 108 of the tubular mesh body 102 of the implant is shown. As described herein, the opening 108 can change (e.g., expand) from an initial size in response to the medical instrument 600 (e.g., catheter) entering and extending through the opening 108 and pushing against the wires 106 forming the opening 108. Upon removal of the medical instrument 600 from the opening 108, the opening 108 can revert back to its initial size from before the medical instrument entered and extended through the opening 108. As shown in FIG. 6, a first portion 602 of the medical instrument 600 is within the lumen 104 of the tubular mesh body, while a second portion 604 of the medical instrument 600 extends through the opening 108 and is external to the lumen 104. When the implant 100 is positioned within a multifurcated zone 300, such a second portion 604 of the medical instrument 600 may extend into a first branch blood vessel 304 and/or subsequent blood vessels connected thereto.

Other aspects of the invention include a method for preventing ischemic stroke by a method of delivering an implantable embolic material diversion device to a multifurcated zone dividing a main blood vessel into at least two branch blood vessels. The methods may include positioning an implant 100 within a multifurcated zone 300. In general, the implant 100 may be introduced, delivered, positioned, and implanted within a multifurcated zone 300 using a delivery device, such as one or more of a guiding catheter, a guiding wire, and a pusher wire. The guiding catheter can be a flexible, small diameter catheter having, for example, a diameter (e.g., outer diameter) in a range of 5 French (Fr) (1.67 mm) to 7 Fr (2.33 mm). The guiding catheter may have lumen in which the implant can be positioned, advanced from, and retracted into. The implant 100 may be introduced by an introducer sheath/guiding catheter combination placed in the femoral artery or groin area of a patient. The implant 100 may be introduced by an introducer sheath/guiding catheter combination placed in the radial artery or forearm (e.g., wrist) area of a patient. In some instances, the implant 100 is guided into the multifurcated zone 300 from an introduction point (e.g., groin or forearm area) with one or more guiding wires (e.g., long, torqueable proximal wire sections with more flexible distal wire sections designed to be advanced within tortuous vessels). Such guiding wires may be visible using fluoroscopy and may be used to first access the multifurcated zone 300, thereby allowing the implant 100 to be advanced over it into the multifurcated zone 300. In some instances, the implant 100 is guided and advanced into the multifurcated zone 300 with one or more pusher wires (e.g., wire sections designed to advance the implant 100 and/or guiding catheter within tortuous vessels).

In some instances, once the implant 100 has accessed the multifurcated zone 300, the implant 100 is advanced and removed from the catheter lumen of the guiding catheter using the pusher wire. While the implant 100 is disposed within the lumen of the guiding catheter, the implant 100 and its tubular body have the radially-compressed position. A user (e.g., a physician) may advance and/or retract the implant 100 several times (e.g., using the pusher wire) to obtain a desirable position of the implant 100 within the multifurcated zone 300 to cause the implant 100 to extend between the main blood vessel 302 and the second branch blood vessel 306 and cover (e.g., block) the inlet 308 to the first branch blood vessel 304. As an example, the user may reposition the implant 100 within the multifurcated zone 300 when the implant 100 is less than 90% advanced out from the guiding catheter and greater than 10% retracted into the guiding catheter. Once the implant 100 is satisfactorily positioned, it can be fully released from the guiding catheter into the multifurcated zone 300 (e.g., by the pusher wire). Upon release, the implant 100 and its tubular mesh body 102 can self-expand from the radially-compressed position toward the fully radially-expanded position. As the implant 100 self-expands (e.g., from the radially-compressed position), an exterior of the tubular mesh body 102 comes into contact with the inner walls of the main blood vessel 302 and the second branch blood vessel 306, thereby slowing and stopping further expansion of the tubular mesh body 102 toward fully radially-expanded position and causing the tubular mesh body 102 to have the radially-expanded shape as described herein. The expansion of the implant 100 and its tubular mesh body 102 is caused by the shape memory nature of the material used to form the implant 100.

In some embodiments, a method of delivering an implant 100 to a multifurcated zone 300 includes advancing the implantable embolic material diversion device, via a delivery device (e.g., guiding catheter, pusher wire, and/or guiding wire), in proximity of the multifurcated zone 300. The method can include releasing the implantable embolic material diversion device from the delivery device (e.g., guiding catheter) into the multifurcated zone (e.g., using the pusher wire). The guiding catheter can have a diameter in a range from 5 Fr (1.67 mm) to 7 Fr (2.33 mm). In some cases, releasing (e.g., fully releasing) the implant 100 from the delivery device into the multifurcated zone 300 causes the tubular mesh body 102 of the implant 100 to expand from a radially-compressed position to a radially-expanded position. In some cases, the method can include at least partially retracting the implant 100 into the delivery device from the multifurcated zone 300, thereby causing at least part (e.g., a proximal end 112) of the tubular mesh body 102 to compress into the radially-compressed position (e.g., inside of a lumen of the delivery device). In some cases, releasing the implant 100 from the delivery device (e.g., guiding catheter) into the multifurcated zone 300 causes one or more of (i) the proximal anchor 114 to anchor at least the proximal end 112 of the tubular mesh body 102 to an inner wall of the main blood vessel 302 on a proximal side 322 of an inlet 308 to a first branch blood vessel 304 and (ii) the distal anchor 118 to anchor at least the distal end 116 of the tubular mesh body 102 to an inner wall of a second branch blood vessel 306 on a distal side 324 of the inlet 308 to the first branch blood vessel 304.

In some embodiments, the method can include performing re-access and/or re-crossing. In some cases, re-access and/or recrossing can include advancing a medical instrument through a particular one of the openings 108 of the tubular mesh body 102 and into the inlet 308 to the first branch blood vessel 304, thereby causing (i) expansion of the particular opening 108 from an initial size and (ii) elastic deformation of a portion of the tubular mesh body 102 adjacent to (e.g., surrounding) the particular opening 108. In some cases, elastic deformation of the portion of the tubular mesh body 102 adjacent to the particular opening 108 can cause sizes of openings 108 included in that portion to compress from their initial size. In some cases, re-access and/or recrossing can include retracting the medical instrument from the particular opening 108 and out of the inlet 308 to the first branch blood vessel 304, whereby the particular opening 108 returns to its initial size when the medical instrument is retracted from the particular opening 108. In some cases, retracting the medical instrument from the particular opening 108 and out of the inlet 308 to the first branch blood vessel 304 can cause sizes of openings 108 included in that portion to return to their initial sizes. Thus, the implant 100 and tubular mesh body 102 enable the medical instrument to access the first branch blood vessel 304 and blood vessels connected thereafter, allowing for the implant 100 to remain positioned within the multifurcated zone 300 when access to the first branch blood vessel 304 and blood vessels connected thereafter is required (e.g., in a medical event such as a medical emergency). As an example, the implant 100 and tubular mesh body 102 enable the medical instrument to access the ICA or vertebral artery and intracranial blood vessels connected thereafter, allowing for the implant 100 to remain positioned within the multifurcated zone 300 when access to the ICA or vertebral artery and subsequent intracranial blood vessels is required.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An implantable embolic material diversion device, comprising:
a tubular mesh body comprising a proximal end and a distal end and defining a lumen between the proximal and distal ends, the tubular mesh body having only a single layer of a plurality of braided wires that form a plurality of openings, each braided wire comprising a shape memory material, and each opening sized to allow a passage therethrough of blood and to prevent the passage therethrough of embolic material having, in every direction of view thereof, at least one straight-line distance between two points on a perimeter of at least one cross-sectional surface in excess of 350 μm.

2. The device of claim 1, wherein each opening is sized to prevent the passage therethrough of embolic material having, in every direction of view thereof, at least one straight-line distance between two points on a perimeter of at least one cross-sectional surface in excess of 200 μm.

3. The device of claim 1, wherein an inscribed circle of each opening comprises a diameter in a range from 180 μm to 400 μm.

4. The device of claim 1, wherein each opening comprises an open cell.

5. The device of claim 1, wherein each opening comprises a polygonal opening.

6. The device of claim 1, wherein a cross-section of each of the plurality of wires has a diameter in a range from 80 μm to 120 μm.

7. The device of claim 1, wherein the plurality of wires number between 24 and 48 wires.

8. The device of claim 1, wherein the shape memory material is a material selected from the group consisting of nitinol, copper-based alloy, and iron-based alloy.

9. The device of claim 1, wherein the shape memory material comprises a biocompatible material.

10. The device of claim 1, wherein the tubular mesh body is self-expandable from a radially-compressed position to a radially-expanded position.

11. The device of claim 10, wherein the radially-compressed position defines a delivery state of the tubular mesh body.

12. The device of claim 10, wherein the radially-expanded position defines an implanted state of the tubular mesh body.

13. The device of claim 1, further comprising a proximal anchor at the proximal end of the tubular mesh body and a distal anchor at the distal end of the tubular mesh body.

14. The device of claim 13, wherein the proximal and distal anchors form a hybrid anchoring structure.

15. The device of claim 13, wherein the proximal anchor comprises a looped anchor structure formed from a subset of the plurality of wires.

16. The device of claim 13, wherein the distal anchor comprises a crowned anchor structure formed from a subset of the plurality of wires.

17. A method of delivering an implantable embolic material diversion device to a multifurcated zone dividing a main blood vessel into at least two branch blood vessels, the method comprising:
advancing the implantable embolic material diversion device, via a delivery device, in proximity of the multifurcated zone, the implantable embolic material diversion device comprising:
a tubular mesh body comprising a proximal end and a distal end and defining a lumen between the proximal and distal ends, the tubular mesh body having only a single layer of a plurality of braided wires that form a plurality of openings, each braided wire comprising a shape memory material, and each opening sized to allow a passage therethrough of blood and to prevent the passage therethrough of embolic material having, in every direction of view thereof, at least one straight-line distance between two points on a perimeter of at least one cross-sectional surface in excess of 350 μm; and
releasing the implantable embolic material diversion device from the delivery device into the multifurcated zone.

18. The method of claim 17, wherein the delivery device comprises a pusher wire and a guiding catheter having a diameter in a range from 5 French (Fr) (1.67 mm) to 7 Fr (2.33 mm).

19. The method of claim 17, wherein releasing the implantable embolic material diversion device from the delivery device into the multifurcated zone causes the tubular mesh body to expand from a radially-compressed position to a radially-expanded position.

20. The method of claim 19, further comprising:
at least partially retracting the implantable embolic material diversion device into the delivery device from the multifurcated zone, thereby causing at least part of the tubular mesh body to compress into the radially-compressed position.

21. The method of claim 17, wherein the implantable embolic material diversion device further comprises a proximal anchor at the proximal end of the tubular mesh body and a distal anchor at the distal end of the tubular mesh body.

22. The method of claim 21, wherein releasing the implantable embolic material diversion device from the delivery device into the multifurcated zone causes (i) the proximal anchor to anchor at least the proximal end of the tubular mesh body to a wall of the main blood vessel on a proximal side of an inlet to a first branch blood vessel and (ii) the distal anchor to anchor at least the distal end of the tubular mesh body to a wall of a second branch blood vessel on a distal side of the inlet to the first branch blood vessel.

23. The method of claim 22, wherein the main blood vessel comprises a proximal portion of a subclavian artery or a brachiocephalic trunk, the first branch blood vessel comprises a vertebral artery, and the second branch blood vessel comprises a distal portion of the subclavian artery.

24. The method of claim 22, wherein the main blood vessel comprises a common carotid artery, the first branch blood vessel comprises an internal carotid artery, and the second branch blood vessel comprises an external carotid artery.

25. The method of claim 17, further comprising:
advancing a medical instrument through a particular one of the openings and into an inlet to a first branch blood vessel, thereby causing (i) expansion of the particular opening from an initial size and (ii) elastic deformation of a portion of the tubular mesh body adjacent to the particular opening; and
retracting the medical instrument from the particular opening and out of the inlet to the first branch blood vessel, whereby the particular opening returns to its initial size when the medical instrument is retracted from the particular opening.
